(19) United States
(12) Patent Application Publication
(10) Pub. No.: US 2009/0192392 A1
(43) Pub. Date: Jul. 30, 2009

(54) Method, system, and apparatus for statistical evaluation of antihypertensive treatment

(76) Inventor: Pedro Mateo Riobo Aboy, Portland, OR (US)

Correspondence Address: ABOY&ASSOCIATES PC | Dr. Mateo Aboy #64040;www.aboypatentlaw.com, 522 SW 5th Ave, Suite 1265, Portland, OR, 97204, US

(21) Appl. No.: 11/756,337

(22) Filed: May 31, 2007

(51) Int. Cl. A61B 5/02

(52) U.S. Cl. 600/485

(57) ABSTRACT

A novel method, system, and apparatus—the RDH Method—for evaluating antihypertensive treatment efficacy across patient populations is disclosed. In accordance to one embodiment, the RDH is a population vector index and graphical method that provides the means for the statistical assessment of antihypertensive treatment reduction, duration, and homogeneity using ambulatory blood pressure monitoring (ABPM). The population RDH was specifically designed as a tool to evaluate and compare blood pressure (BP) coverage offered by antihypertensive drugs over 24 h in populations. In accordance to one embodiment, the population RDH is a three-component vector index that incorporates information about the reduction, duration, and homogeneity of antihypertensive treatment, as well as their statistical significance over the 24 h period. In the preferred embodiment, the population RDH components quantify: 1) the total number of statistical significant BP reductions, 2) the maximum number of consecutive statistical significant reductions, and 3) the maximum number of consecutive non-significant reductions over the 24 hours, respectively; and reports two population graphs that characterize the effect of the treatment. The output of the RDH index can be used in clinical trials to characterize the effects of antihypertensive medications, and in clinical practice to guide antihypertensive treatment.

### CROSS REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND

1. Field of Invention

This invention relates to evaluation of antihypertensive treatments, specifically it relates to methods, system or apparatus for statistical evaluation of antihypertensive treatment reduction, duration, homogeneity, and efficacy.

2. Prior Art

Hypertension is a serious chronic disorder that affects over 1 billion people worldwide. In the United States alone, hypertension affects over 55 million Americans. Chronic hypertension is associated with increased morbidity and mortality. When left untreated, the prolonged elevation of BP produces serious end organ damage, including myocardial infarction, heart failure, angina pectoris, left ventricular hypertrophy, prior coronary revasculazation, kidney disease, stroke, peripheral arterial disease, transient ischemic attack, and retinopathy [1,2].

The ultimate objective in treating hypertension is to reduce cardiovascular and renal morbidity and mortality. A secondary goal is to accomplish BP control without decreasing the quality of life with the drugs employed. The incidence of mortality and morbidity decreases significantly when hypertension is diagnosed early and property treated with adequate antihypertensive treatments. Drug therapy can reduce both BP and the risk of long-term complications. However, since antihypertensive treatment is not curative it is necessary to continue treatment indefinitely. This presents one of the first challenges in the treatment of hypertension. Despite the potential serious harm of elevated BP, hypertension is usually asymptomatic until after end-organ damage. Thus, since elevated BP often doesn't cause discomfort, many people with essential hypertension do not get treated. Even when hypertension is property diagnosed, compliance to treatment is a significant problem. As a consequence, single dose antihypertensive drugs with 24-h duration of action are preferred to treat essential hypertension. Single-dose treatments help promote compliance. However, it is important that single-dose drugs intended for monotherapy regimes have an effective 24-h duration of action. Single-dose treatments must provide adequate BP control for 24-h to be useful as antihypertensive drugs.

The introduction of ambulatory blood pressure monitoring (ABPM) has enabled clinicians and researchers to provide better diagnosis and treatment of hypertension. ABPM has drastically improved the ability to assess the efficacy of antihypertensive treatment in clinical studies and in medical practice [3-5]. ABPM offers significant advantages over clinic sphygmomanometric readings as a toot to property diagnose, treat hypertension, and evaluate antihypertensive treatment. These include: 1) ABPM is characterized by higher reproducibility, 2) it is not subject to observer bias and white-coat effect, 3) it enables practitioners to test the effectiveness of a given antihypertensive drug in daily life conditions, and 4) can be used to estimate the pharmacodynamics of antihypertensive drugs [6-8]. ABPM enables clinicians and researchers to obtain accurate and reproducible data on the circadian pattern of BP. With ABPM, multiple automatic measurements of BP are obtained at specific intervals throughout the day. However, despite all the advantages of ABPM over casual BP readings, the analytic techniques and indices used to analyze the ABPM recordings are not taking full advantage of the ABPM benefits and the current indices used to evaluate antihypertensive drugs—the trough:peak ratio (TP) and the smoothness index (SI)—have significant limitations and do not provide a complete and accurate characterization of the treatment [7,9-14].

Despite its significant benefits, antihypertensive treatment is not curative, must be life-long, and is very costly. In 1998, $108 billion in health care spending was attributed to hypertension in the US. The medical, economic, and human costs of untreated and inadequately controlled high blood pressure are enormous [15-34].

A previous method for evaluating and treating hypertension has been proposed in U.S. Pat. No. 6,632,180 (2003) and U.S. Pat. No. 6,595,926 to Laragh J. H. This method is manual, not based on statistical inference, is not designed to work with ABPM, and does not account for chronopharmatheucal effects. Other methods for evaluating antihypertensive treatment based on ABPM have been described in the scientific literature and are public domain (described below).

As mentioned earlier, ABPM has drastically improved the ability to assess the efficacy of antihypertensive treatment in clinical studies and in medical practice [3-5]. Consequently, state-of-the art diagnosis, treatment, and evaluation of antihypertensive therapy is based on ABPM. Despite the significant advantages of ABPM, the current indices and available techniques for ABPM analysis are limited. For instance, duration and homogeneity of antihypertensive drugs are commonly quantified by computation of the TP and SI [7, 9-14]. Normally both the TP and the SI are calculated from ABPM recordings obtained from individual subjects and have important limitations when applied to populations [11,35-38]. Evaluation of antihypertensive treatment in populations is often carried out by calculating these individual indices for each of the subjects and providing summarizing statistics about the population such as the mean and median. However, the lack of a well-defined population index (i.e. an index specifically developed to analyze population data as opposed to individual ABPM recordings) has resulted in methodological inconsistencies regarding the description of antihypertensive drug effect at the population level. Currently, researchers do not follow a standardized methodology to conduct and report results on populations. This limits significantly the comparability and reproducibility of results involving the evaluation of antihypertensive treatment, and prevents the use of evidence-based medicine by practitioners. Additionally, given the limited information provided by these indices to characterize antihypertensive therapy, it is normally required to perform additional statistical analysis of the ABPM recordings to characterize the antihypertensive therapy. Leading experts in the research community have pointed out the limitations of the TP index. For instance, a leading researcher has stated “although it is widely employed, this index has a lot of limitations” [12], another leading scientist also pointed out that “the lack of a specific methodology for estimating the TP initially led to considerable confusion in the literature with each investigator taking a unique approach to obtaining a TP value”, warned that “there are still important methodological issues that have yet to be resolved”, and recommended that “the use of the TP should be reconsidered and probably abandoned” [35]. The SI index was introduced to overcome some of the limitations of the TP index and was shown to correlate with end-organ damage [39]. However, the SI is also limited and leading researchers have concluded that “overall, neither index has been proven to offer definitive superiority” [11], and cautioned of the intrinsic limitations of these techniques to evaluate and compare antihypertensive treatment by stating that “any inference on the clinical superiority of one particular treatment regime agent over another based on a higher TP, MER or SI remains largely speculative in nature” [13].

Both the TP and SI have established definitions in the literature for their evaluation on individual subjects. However, most studies involving assessment of antihypertensive effects are based on populations, and require researchers to report an index to characterize the population or the specific antihypertensive treatment under study. The typical approach to solve this problem has been to evaluate the TP and SI for each individual subject in the sample population under study, and to use summarizing statistics such as the mean or median to report results to characterize the population. Another approach has been to adapt and/or redefine individual indices so that they can be calculated directly from the population, leading to the concept of population indices (i.e. indices calculated directly on the population) versus individual indices (i.e. indices calculated on individual subjects).

Before the introduction of the SI in 1998, the TP was the only established index used for assessment of antihypertensive treatment. Initially, the characterization of populations was done by reporting the mean of the individual indices, that is, the mean of the TPs [9, 40]. However, since the TP does not follow a normal distribution Omboni proposed to characterize the population by providing the median of the individual TPs [12,41,42]. In addition to the median of the individual TPs, it was later proposed by Meredith, Stergiou, and Mancia to provide also a measure of dispersion such as the range of the individual TP values [11], the inter-quartile range [14], or the 5th and 95th percentiles [43,44]. This methodology was not universally adopted by the research community, and recent studies have used the mean to characterize the population and in some cases the mean of responders [45,46]. Additionally, there is another methodology proposed by Stewart that consists in calculating the so-called population TP, as the ratio of the mean of all the individual troughs and the mean of all the individual peaks [47, 48].

In the case of the SI, since its introduction it was reported to follow a normal distribution

. As a consequence, it is most commonly reported on populations by providing the mean of the individual SIs and the standard error [12,14,39,43-46].

Consequently, the design and development of a well-designed method to statistically characterize and compare antihypertensive treatment in individuals and populations is an important problem identified by the research community and practitioners.

### SUMMARY

The present invention discussed herein provides a novel method, system, and apparatus—the RDH Method—for evaluating antihypertensive treatment efficacy across patient populations. In accordance to one embodiment, the RDH is a population vector index and graphical method that provides the means for the statistical assessment of antihypertensive treatment reduction, duration, and homogeneity using ambulatory blood pressure monitoring (ABPM). The population RDH was specifically designed as a tool to evaluate and compare blood pressure (BP) coverage offered by antihypertensive drugs over 24 h in populations. In accordance to one embodiment, the population RDH is a three-component vector index that incorporates information about the reduction, duration, and homogeneity of antihypertensive treatment, as well as their statistical significance over the 24 h period. In the preferred embodiment, the population RDH components quantify: 1) the total number of statistical significant BP reductions, 2) the maximum number of consecutive statistical significant reductions, and 3) the maximum number of consecutive non-significant reductions over the 24 hours, respectively; and reports two population graphs that characterize the effect of the treatment. The output of the RDH index can be used in clinical trials to characterize the effects of antihypertensive medications, and in clinical practice to guide antihypertensive treatment.

### DRAWINGS/FIGURES

FIG. 1 shows an example of the graphical results detailing the antihypertensive effect on the patient population according to one embodiment of the said RDH method proposed (Analysis based on SBP). Comparison of the RDHp on the treated versus non-treated groups. (a) Nonparametric RDHp. (b) Parametric RDHp. (c) Nonparametric RDHp . . . 20

FIG. 2 shows an example of the graphical results detailing the antihypertensive effect on the patient population according to one embodiment of the said RDH method proposed (Analysis based on DBP). Comparison of the RDHp on the treated versus non-treated groups. (a) Nonparametric RDHp. (b) Parametric RDHp. (c) Nonparametric RDHp. Analysis based on DBP . . . 21

FIG. 3 shows an example of the graphical results detailing the antihypertensive effect on the patient population and individuals according to one embodiment of the said RDH method proposed. Nonparametric RDH population plot for treated group (analysis based on SBP). The top plot shows the individual RDH sequence for each subject. In this plot a gray square indicates an statistically significant reduction, the absence of a square corresponds to a non-significant BP reduction, and a white square denotes a time category where no data was available to perform the statistical test. This graph complements the RDHp plot by displaying the RDH corresponding to each individual subject in the population under study. The bottom plot in this graph shows the proportion of statistically significant reductions in each category, and whether the population RDH resulted in a statistically significant reduction (gray circle) or in a non-significant reduction (white circle) . . . 22

FIG. 4 shows an example of the graphical results detailing the antihypertensive effect on the patient population and individuals according to one embodiment of the said RDH method proposed. Nonparametric RDH population plot for non-treated group (analysis based on SBP). The top plot shows the individual RDH sequence for each subject. In this plot a gray square indicates an statistically significant reduction, the absence of a square corresponds to a non-significant BP reduction, and a white square denotes a time category where no data was available to perform the statistical test. This graph complements the RDHp plot by displaying the RDH corresponding to each individual subject in the population under study. The bottom plot in this graph shows the proportion of statistically significant reductions in each category, and whether the population RDH resulted in a statistically significant reduction (gray circle) or in a non-significant reduction (white circle). This visualization tools enables researchers to quickly compare treatment or populations. As expected, the number of statistical significant reductions is much lower in the non-treated group than in the treated group . . . 23

### DETAILED DESCRIPTION—PREFERRED EMBODIMENT

Given an individual ABPM recording, we denote each of the time categories by an index k, where {k}ₖ₌₁^{K}. For the purposes of describing this particular embodiment we will assume we have 24 categories (K=24) corresponding to 24 h. Let Lₖ represent number of BP samples in the k-th class at baseline. In general, the dimension of vectors from different classes is not equal, that is, Lₖ≠Lⱼ where k and j denote the index of the k-th and j-th class. Analogously, let Lₖ¹ number of BP samples in the k-th class after treatment. In general, Lₖ≠Lₖ¹, that is, the dimension of the vector before treatment corresponding to the k-th category is not necessarily equal to the dimension of the vector after treatment corresponding the same category. Let x denote vector containing the individual BP values before treatment, and let x_{k,i} denote the i-th sample belonging to time category k,

$${x}_{1} = ( {x}_{1 , 1} , {x}_{1 , 2} , … , {x}_{1 , {L}_{1}} ) \\ {x}_{2} = ( {x}_{2 , 1} , {x}_{2 , 2} , … , {x}_{2 , {L}_{2}} ) \\ ⋮ \\ {x}_{24} = ( {x}_{24 , 1} , {x}_{24 , 2} , … , {x}_{24 , {L}_{24}} ) \qquad ( 1 )$$

The vector y containing the BP values after treatment for the same subject is defined analogously,

$${y}_{1} = ( {y}_{1 , 1} , {y}_{1 , 2} , … , {y}_{1 , {L}_{1}^{′}} ) \\ {y}_{2} = ( {y}_{2 , 1} , {y}_{2 , 2} , … , {y}_{2 , {L}_{2}^{′}} ) \\ ⋮ \\ {y}_{24} = ( {y}_{24 , 1} , {y}_{24 , 2} , … , {y}_{24 , {L}_{24}^{′}} ) \qquad ( 2 )$$

Let x̅ₖ, y̅ₖ denote the sample mean of the ABPM vector corresponding to the k-th category before and after treatment,

$${\overline{x}}_{k} = {\sum }_{i = 1}^{{L}_{k}} \frac{{x}_{k , i}}{{L}_{k}} , {\overline{y}}_{k} = {\sum }_{i = 1}^{{L}_{k}^{′}} \frac{{y}_{k , i}}{{L}_{k}^{′}} ,$$

and let x̅, y̅ be vectors containing sample means before and after treatment,

x̅=( x̅₁, x̅₂, . . . , x̅₂₄)

y̅=( y̅₁, y̅₂, . . . , y̅₂₄) (3)

The vector containing the class-by-class differences is denoted as d,

$$\begin{matrix}d = \overline{x} - \overline{y} \\ = ( {\overline{x}}_{1} , {\overline{x}}_{2} , … , {\overline{x}}_{24} ) - ( {\overline{y}}_{1} , {\overline{y}}_{2} , … , {\overline{y}}_{24} ) \\ = ( {d}_{1} , {d}_{2} , … , {d}_{24} )\end{matrix} \qquad ( 4 )$$

In order to define the population RDH we define xₖ^{j} to be xₖ for subject j. Given J subjects in the population under study, we have

$${x}_{1}^{1} = ( {x}_{1 , 1}^{1} , {x}_{1 , 2}^{1} , … , {x}_{1 , {L}_{1 , 1}}^{1} ) \\ ⋮ \\ {x}_{24}^{1} = ( {x}_{24 , 1}^{1} , {x}_{24 , 2}^{1} , … , {x}_{24 , {L}_{24 , 1}}^{1} ) \\ {x}_{1}^{2} = ( {x}_{1 , 1}^{2} , {x}_{1 , 2}^{2} , … , {x}_{1 , {L}_{1 , 2}}^{2} ) \\ ⋮ \\ {x}_{24}^{2} = ( {x}_{24 , 1}^{2} , {x}_{24 , 2}^{2} , … , {x}_{24 , {L}_{24 , 2}}^{2} ) \\ ⋮ \\ {x}_{1}^{J} = ( {x}_{1 , 1}^{J} , {x}_{1 , 2}^{J} , … , {x}_{1 , {L}_{1 , J}}^{J} ) \\ ⋮ \\ {x}_{24}^{J} = ( {x}_{24 , 1}^{J} , {x}_{24 , 2}^{J} , … , {x}_{24 , {L}_{24 , J}}^{J} ) \qquad ( 5 )$$

the vector yₖ^{j} is defined analogously.

The population RDH can be calculated based on parametric or nonparametric statistics. The advantage of the nonparametric RDH is that it minimizes the number of assumptions made.

The parametric population RDH is obtained as follows: For each category k, the population RDH takes as an input the set of before {xₖ^{j}}ⱼ₌₁^{J} and post-treatment {yₖ^{j}}ⱼ₌₁^{J} ABPM recordings, and generates a three component vector index according to the following algorithm:

Calculate the mean of each category k for each subject j before and after the treatment

$${\overline{x}}_{k}^{j} = {\sum }_{i = 1}^{{L}_{k , j}} \frac{{x}_{k , i}^{j}}{{L}_{k , j}} \\ {\overline{y}}_{k}^{j} = {\sum }_{i = 1}^{{L}_{k , j}^{′}} \frac{{y}_{k , i}^{j}}{{L}_{k , j}^{′}} \qquad ( 6 )$$

Create population composites of category k, before xₖ and after treatment yₖ,

$$\begin{matrix}{x}_{k} = ( {\overline{x}}_{k}^{1} , {\overline{x}}_{k}^{2} , … , {\overline{x}}_{k}^{j} , ⋮ , {\overline{x}}_{k}^{J} ) & ( 7 ) \\ {y}_{k} = ( {\overline{y}}_{k}^{1} , {\overline{y}}_{k}^{2} , … , {\overline{y}}_{k}^{j} , ⋮ , {\overline{y}}_{k}^{J} ) & ( 8 )\end{matrix}$$

Create vector containing the BP differences,

dₖ=xₖ−yₖ=( x̅ₖ¹− y̅ₖ¹, x̅ₖ²− y̅ₖ², . . . , x̅ₖ^{J}− y̅ₖ^{J})=(d₁, d₂, . . . , d_{J}) (9)

Perform a paired-sample t test to test if the mean BP reduction in category k is greater than zero,

$$\begin{matrix}{t}_{k} = \frac{{\overline{d}}_{k}}{{\hat{se}}_{{\overline{d}}_{k}}} = \frac{{\sum }_{j = 1}^{J} \frac{{\overline{x}}_{k}^{j} - {\overline{y}}_{k}^{j}}{J}}{\sqrt{\frac{{s}_{{d}_{k}}^{2}}{J}}} & {s}_{{d}_{k}}^{2} = \sqrt{{\sum }_{j = 1}^{J} \frac{{( {d}_{j} - \overline{d} )}^{2}}{J - 1}}\end{matrix} \qquad ( 10 )$$

that is, for each category k, k=1, . . . , 24, we assess the statistical significance of of the mean BP reduction by dividing the mean BP difference d̅ₖ for category k over its standard error ŝê _{d̅}_{ₖ}.

We define the population RDH vector as RDH=(c₁, c₂, c₃) where
c₁=Total number of statistically significant reductions
c₂=Maximum number of consecutive statistically significant reductions
c₃=Maximum number of consecutive statistically non-significant reductions

Since in general J>30, the t distribution approximates the Normal distribution, and the threshold of 1.645 from the Normal distribution can be used to establish statistical significance.

The nonparametric population RDH is based on bootstrap to estimate the probability density function of the mean BP differences for category k across the population and perform a nonparametric test [49]. The nonparametric population RDH takes as an input the set of before {xₖ^{j}}ⱼ₌₁^{J} and {yₖ^{j}}ⱼ₌₁^{J} ABPM recordings and generates a three component vector index according to the following algorithm

Create Vector vector containing the BP differences,

dₖ=xₖ−yₖ=( x̅ₖ¹− y̅ₖ¹, x̅ₖ²− y̅ₖ², . . . , x̅ₖ^{J}− y̅ₖ^{J}) (11)

Note that even though the probability model of xₖ and yₖ follows the two-sample model, the probability model for the inter-population RDH follows a one-sample model,

Tₖ→dₖ=(d_{k,1}, d_{k,2}, . . . , d_{k,J})

where as previously defined d_{k,j}= x̅ₖ^{j}− y̅ₖ^{j} and Tₖ is the distribution function for category k.

Calculate the statistic of interest from dₖ, \hat{θ}ₖ=s(dₖ), which in this case is the mean BP reduction d̅.

$${\hat{θ}}_{k} = s ( {d}_{k} ) = {\sum }_{j = 1}^{J} \frac{{d}_{k , j}}{J} \qquad ( 12 )$$

Use the empirical distribution \hat{T}ₖ to obtain bootstrap samples dₖ*=(d_{k,1}*, d_{k,2}*, . . . , d_{k,J}*) by random sampling of \hat{T}ₖ

\hat{T}ₖ→dₖ*=(d_{k,1}*,d_{k,2}*, . . . ,d_{k,J}*)

from which we can calculate bootstrap replications of the statistic of interest \hat{θ}ₖ=s(dₖ*) to estimate the probability distribution \hat{θ}ₖ*.

Use the histogram of \hat{θ}ₖ*(b), b=1, 2, . . . , B as an estimate of the probability density function of the mean BP differences for category k across the population. The bootstrap confidence intervals for the population BP reduction in class k are obtained as

\hat{θ}ₖ_{ₗₒ}=100·α^{th} percentile of \hat{θ}ₖ*'s distribution

\hat{θ}ₖ_{ₗₒ}=100·(1−α)^{th} percentile of \hat{θ}ₖ*'s distribution (13)

If this interval contains zero, it cannot be assumed with (1−2α) confidence that the parameters of the two populations are statistically different.

Define the nonparametric population RDH vector as RDH=(c₁, c₂, c₃) analogous to the parametric case.

### Operation: The Following Description Exemplifies how to Interpret the Results of the Preferred Embodiment of the Method or System when used to Analyze Population ABPM Data in Order to Statistically Evaluate an Antihypertensive Treatment and its Relationship to Prior Art—Tp And Si—

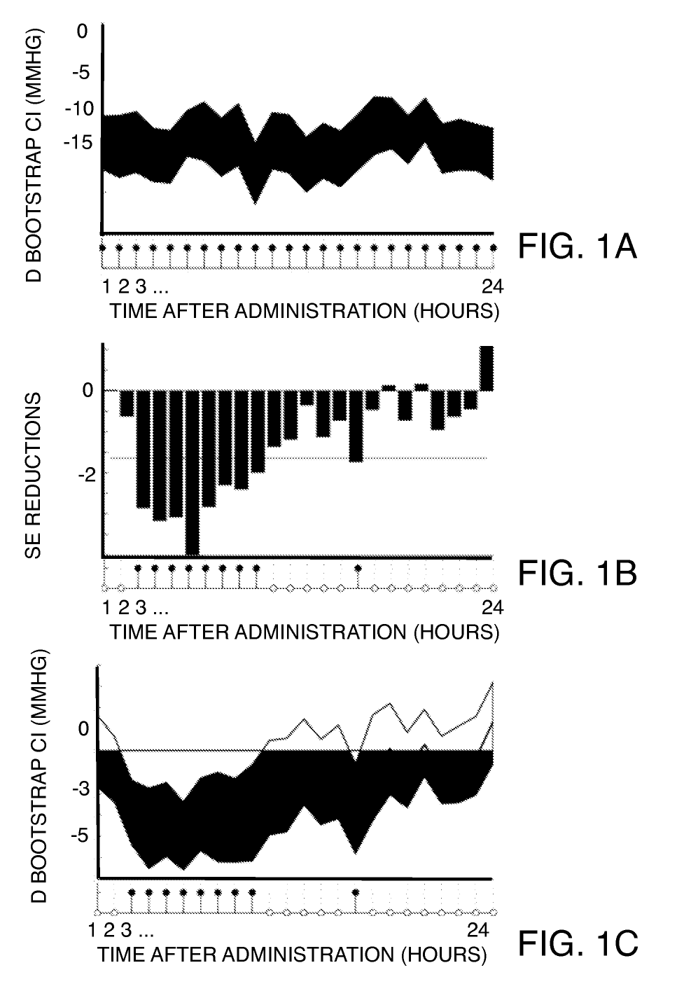

FIG. 1 shows a comparison of a treated group versus a non treated group based on the population RDH (RDHp) described in this paper. In FIG. 1, the RDH index was computed from analysis of the SBP population data. These figures show the results using the parametric (FIG. ??-1(b)) and the nonparametric (FIG. 1(a)-1(c)) versions of the RDH index. Note that both tests lead to the same results. In FIG. ??-1(b) the top plot shows the BP reductions normalized in units of standard errors, the bottom plot shows the statistical (grey) and nonstatistical (white) reductions. In FIG. 1(a)-1(c) the top shows the mean BP reduction and the Bootstrap confidence intervals for each time category. In the case of the treated group, RDHp=(24,24,0), which indicates that there were statistically significant reductions in all the 24 time categories. Based on this RDHp value we can conclude that treatment is a drug with a 24-h duration of action. Furthermore, the RDHp plot shows the estimated confidence intervals and the estimated mean BP reduction. Based on the RDHp plot we can also state that the treatment induced a mean BP reduction of 15 mmHg approximately. The confidence intervals indicate that the reduction is homogeneous across the 24-h period. On the other hand, we see that in the case of the non-treated subjects, RHDp=(9,8,8), that is, there were 9 statistically significant reductions, 8 consecutive statistically significant reductions, and 8 consecutive non-significant reductions. Note that the graph of the RDH index clearly shows that all the statistical significant reductions occurred between hour 3 and hour 10 after waking up. This result suggests that there is an “ABPM Effect” in the first 10 hours of ABPM [50]. The RDHp plot indicates that the mean reduction due to this ABPM effect is approximately 3 mmHg.

Additionally, the RDHp can be used to test the effectiveness of a given antihypertensive treatment on a specific population by comparing the upper confidence interval against a threshold different from zero. For instance, the RDHp can be used to test the number of statistical significant and effective reductions by comparing the confidence interval against a 5 mmHg threshold. Even without performing the statistical test, the current nonparametric RDHp graph showing the confidence intervals can be used for this purpose. In FIG. 1(a), for instance, we can see that if the test threshold were changed from 0 to −7 mmHg, all the reductions would still be statistical significant. Thus, we can conclude that this treatment not only induces statistical significant reductions across the 24 h period, but also that this reductions are all effective against a −7 mmHg threshold. Note that if we were to apply the same criteria to the non-treated group (FIG. 1(c)), none of the reductions would come out statistical significant, indicating non-effectiveness.

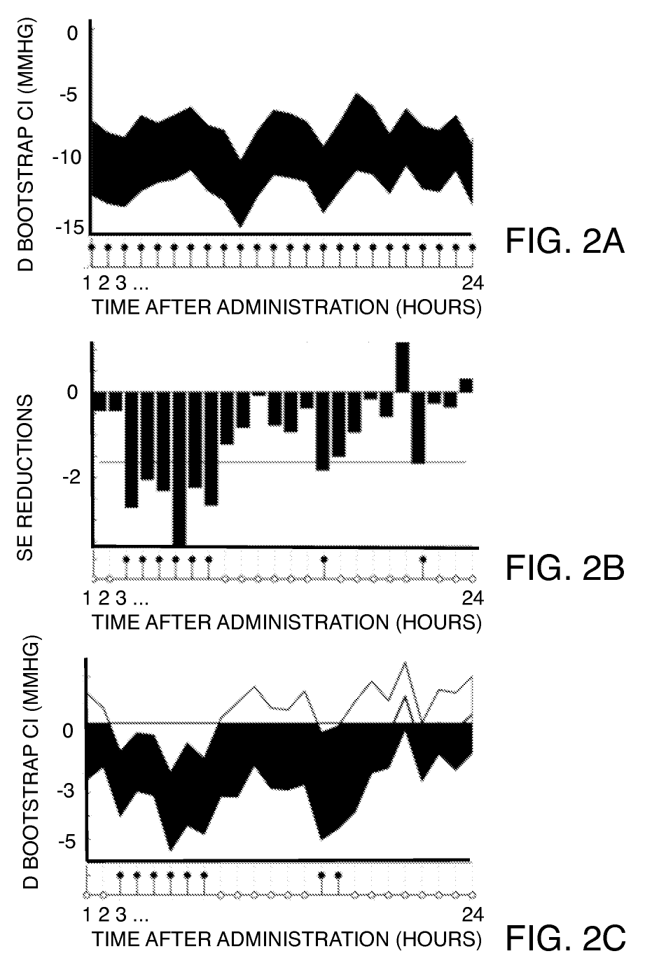

FIG. 2 shows a comparison of the treated versus the non-treated groups based on the RDHp computed from analysis of the DBP population data. In the treated group, RDHp=(24,24,0), which indicates that there were statistically significant reductions in all the 24 time categories. Based on this RDHp value we can conclude that treatment also induces an statistically significant reduction on the DBP of 24-h duration. In the case of the non treated group, RHDp=(8,6,8). From the RDHp graph we can see that the mean BP reduction is lower in the DBP than SBP. This difference is approximately 5 mmHg in the treated group (15 mmHg vs 10 mmHg approximately).

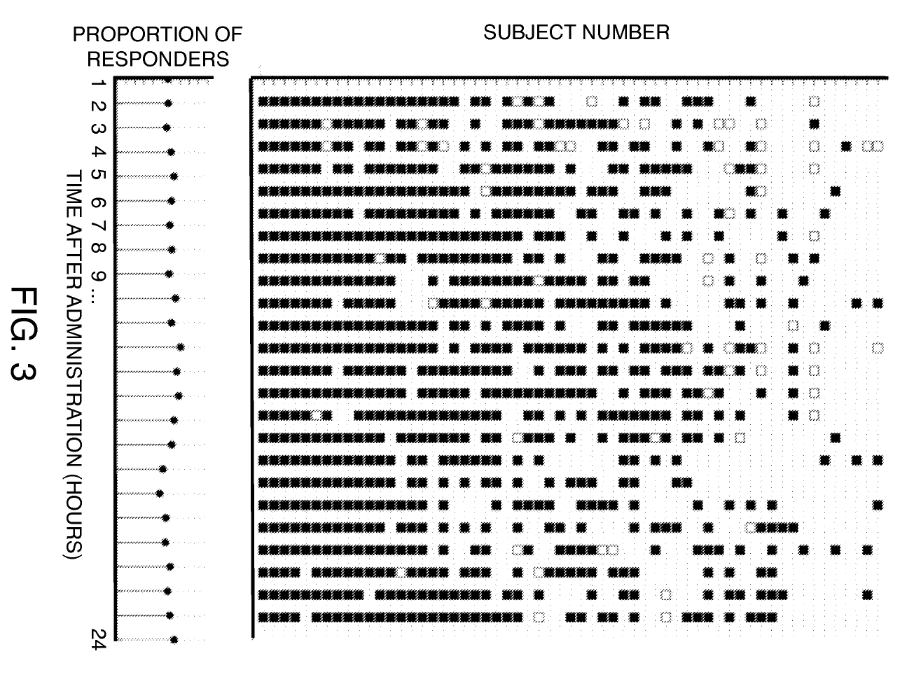

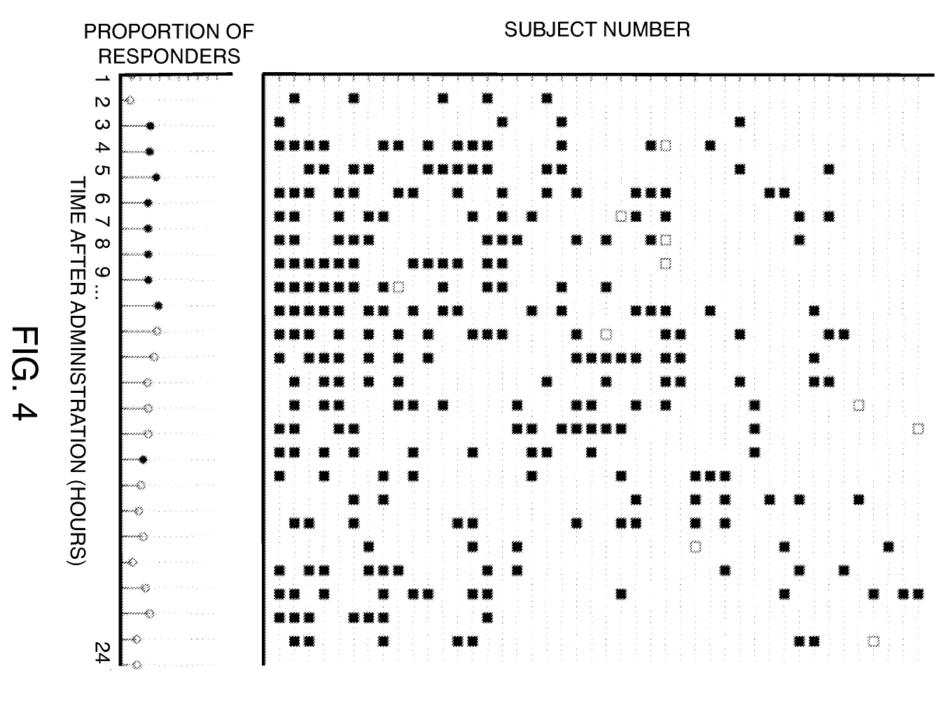

FIG. 3 and FIG. 4 show a graphical representation of the individual RDHs, and a comparison between the treated and the non treated groups on SBP using the nonparametric RDH. The top plot shows the individual RDH sequence for each subject. In this plot a gray square indicates an statistically significant reduction, the absence of a square corresponds to a non-significant BP reduction, and a white square denotes a time category where no data was available to perform the statistical test. This graph complements the RDHp plot by displaying the RDH corresponding to each individual subject in the population under study. The bottom plot in this graph shows the proportion of statistically significant reductions in each category, and whether the population RDH resulted in a statistically significant reduction (gray circle) or in a non-significant reduction (white circle). This graphical representation enables researchers to immediately identify the non-responder subjects (i.e. subjects for whom the antihypertensive treatment did not induced statistically significant reductions across the 24-h period).

What is claimed is:

1. A method for evaluating antihypertensive treatment and guiding antihypertensive therapy using parametric statistical inference, comprising:
(a) obtaining and analyzing two synchronized ABPM recordings: pre-treatment and post-treatment
(b) calculating the mean of each category k for each subject j before and after the treatment
$${\overline{x}}_{k}^{j} = {\sum }_{i = 1}^{{L}_{k , j}} \frac{{x}_{k , i}^{j}}{{L}_{k , j}} \\ {\overline{y}}_{k}^{j} = {\sum }_{i = 1}^{{L}_{k , j}^{′}} \frac{{y}_{k , i}^{j}}{{L}_{k , j}^{′}} \qquad ( 14 )$$
(c) creating population composites of category k, before xₖ and after treatment yₖ,
xₖ=( x̅ₖ¹, x̅ₖ², . . . , x̅ₖ^{j}, [symbol] , x̅ₖ^{J}) (15)
yₖ=( y̅ₖ¹, y̅ₖ², . . . , y̅ₖ^{j}, [symbol] , y̅ₖ^{J}) (16)
(d) creating a vector containing the BP differences,
dₖ=xₖ−yₖ=( x̅ₖ¹− y̅ₖ¹, x̅ₖ²− y̅ₖ², . . . , x̅ₖ^{J}− y̅ₖ^{J})=(d₁,d₂, . . . ,d_{J}) (17)
(e) performing a paired-sample t test to test if the mean BP reduction in category k is greater than zero,
$$\begin{matrix}{t}_{k} = \frac{{\overline{d}}_{k}}{{\hat{se}}_{{\overline{d}}_{k}}} = \frac{{\sum }_{j = 1}^{J} \frac{{\overline{x}}_{k}^{j} - {\overline{y}}_{k}^{j}}{J}}{\sqrt{\frac{{s}_{{d}_{k}}^{2}}{J}}} & {s}_{{d}_{k}}^{2} = \sqrt{{\sum }_{j = 1}^{J} \frac{{( {d}_{j} - \overline{d} )}^{2}}{J - 1}}\end{matrix} \qquad ( 18 )$$
for each category k, k=1, . . . , 24, assessing the statistical significance of of the mean BP reduction by dividing the mean BP difference d̅ₖ for category k over its standard error ŝê _{d̅}_{ₖ}.
(f) defining the parametric embodiment of the population RDH vector as RDH=(c₁, c₂, c₃) where
c₁=Total number of statistically significant reductions
c₂=Maximum number of consecutive statistically significant reductions
c₃=Maximum number of consecutive statistically non-significant reductions

2. A method for evaluating antihypertensive treatment and guiding antihypertensive therapy using nonparametric statistical inference, comprising:
(a) creating a vector containing the BP differences,
dₖ=xₖ−yₖ=( x̅ₖ¹− y̅ₖ¹, x̅ₖ²− y̅ₖ², . . . , x̅ₖ^{J}− y̅ₖ^{J}) (19)
(b) calculating the statistic of interest from dₖ, \hat{θ}ₖ=s(dₖ), which in this case is the mean BP reduction d̅,
$${\hat{θ}}_{k} = s ( {d}_{k} ) = {\sum }_{j = 1}^{J} \frac{{d}_{k , j}}{J} \qquad ( 20 )$$
(c) analyzing the empirical distribution \hat{T}ₖ to obtain bootstrap samples dₖ*=(d_{k,1}*,d_{k,2}*, . . . , d_{k,J}*) by random sampling of \hat{T}ₖ
\hat{T}ₖ→dₖ*=(dₖₘ₁*,d_{k,2}*, . . . ,d_{k,J}*)
and calculating bootstrap replications of the statistic of interest \hat{θ}ₖ=s(dₖ*) to estimate the probability distribution \hat{θ}ₖ*,
(d) analyzing the histogram of \hat{θ}ₖ*(b), b=1,2, . . . , B as an estimate of the probability density function of the mean BP differences for category k across the population; the bootstrap confidence intervals for the population BP reduction in class k are obtained as
\hat{θ}ₖ_{ₗₒ}=100·α^{th} percentile of \hat{θ}ₖ*'s distribution
\hat{θ}ₖ_{ₗₒ}=100·(1−α)^{th} percentile of \hat{θ}ₖ*'s distribution (21)
and concluding that if this interval contains zero, it cannot be assumed with (1−2α) confidence that the parameters of the two populations are statistically different,
(e) defining the nonparametric population RDH vector as RDH=(c₁, c₂, c₃) analogous to the parametric case.

3. The method of claim 1, further comprising generating a results image illustrating the antihypertensive treatment effects, including a user-specified confidence interval of the reduction in hypertension on the patient population across the 24-hours.

4. The method of claim 1, further comprising generating a results image the antihypertensive treatment effects for each individual in the same plot, including the statistical significance of the reduction and the proportion of subjects where the treatment results in statistical significant reduction across the 24-hours.

5. The method of claim 2, further comprising generating a graph illustrating the antihypertensive treatment effects, including a user specified confidence interval of the reduction in hypertension on the patient population across the 24-hours.

6. The method of claim 2, further comprising generating a results image illustrating the antihypertensive treatment effects for each individual in the same plot, including the statistical significance of the reduction and the proportion of subjects where the treatment results in statistical significant reduction across the 24-hours.

7. A method for evaluating antihypertensive treatment reduction, duration, homogeneity, efficacy, effectiveness comprising the evaluation of hour-by-hour (overlapping and nonoverLapping) pre-treatment and post-treatment ABPM recordings using parametric or nonparametric statistical inference techniques to determine whether the blood pressure reduction was due to chance or to the treatment working as intended.

8. The method of claim 7, further comprising analyzing ABPM recording corresponding to different antihypertensive treatments to determine chronopharmacodynamical bioequivaLence between treatments.

9. The method of claim 1 or claim 2, wherein the threshold used for establishing statistical significance is user-specified to enable efficacy characterization.

10. A machine or system, comprising hardware and software that implements the methods of claim 1.

11. A machine or system, comprising hardware and software that implements the methods of claim 2.

12. A machine or system, comprising hardware and software that implements the methods of claim 3.
